(19) United States
(12) Patent Application Publication
(10) Pub. No.: US 2024/0342275 A1
(43) Pub. Date: Oct. 17, 2024

(54) SYSTEM AND METHOD FOR ANTIBODY ACTIVATORS IN AN AQUEOUS IODINE SOLUTION

(71) Applicant: Terence Francis Mullins, Magog (CA)

(72) Inventor: Terence Francis Mullins, Magog (CA)

(21) Appl. No.: 18/750,786

(22) Filed: Jun. 21, 2024

Related U.S. Application Data
(63) Continuation-in-part of application No. PCT/IB2022/056854, filed on Jul. 25, 2022.
(60) Provisional application No. 63/292,479, filed on Dec. 22, 2021.

(51) Int. Cl. A61K 39/385; A61K 39/00; C02F 1/68

(52) U.S. Cl. CPC A61K 39/385; C02F 1/685; A61K 2039/60; C02F 2201/005; C02F 2301/043

(57) ABSTRACT

A composition, method and system are provided for an antibody activator. The composition includes an antigen and iodine. The method for preparing the composition includes providing an antigen, providing an iodine carrier for the antigen, and combining the antigen and the iodine carrier. The system for preparing the composition for antibody activation includes an airless reservoir to contain a volume of iodine, an injection device for injecting an antigen into the reservoir containing the iodine, and a pump for pumping the antigen and iodine mixture from the reservoir to a delivery device.

The present application is a continuation in part of PCT application PCT/IB2022/056854, filed on Jul. 25, 2022, presently pending. PCT application PCT/IB2022/056854 in turn claimed priority to U.S. provisional 63/292,479 filed on Dec. 22, 2021, presently expired. The contents of each application is hereby incorporated by reference.

### FIELD

The present disclosure relates to immune system activators, and more particularly to a system and method for delivery of antibody activators in an aqueous iodine solution.

### BACKGROUND

Technologies have evolved to assist or teach the human immune system to recognize viral or bacterial pathogens in order to create antibodies and provide an immune response. Vaccines have formed a major part of this advancement, in which an antigen, usually a part of the pathogen, is introduced into a recipient in order to generate an immune response, primarily by antibodies that recognize the antigen.

At the turn of the twentieth century scientists produced the first vaccines using attenuated virus. The structure of the antigenic material utilized in vaccines has changed over the last century. The delivery solutions for the inert materials that form the antigen have also been evolving.

With an increase of new viruses, it has become difficult for traditional vaccines to keep up with the new strains and variants effectively or quickly enough. New methods have been employed to produce synthetic versions of viral and bacterial proteins, produced in vitro or produced in vivo using injected DNA and RNA, to stimulate the immune system to produce antibodies.

Traditional delivery for vaccines is muscular injection performed by a certified administer. Due to high chemical and material concentrations these products could be lethal if injected directly into the blood stream. Current methods require significant mobilization of human resources to both administer and the patient's ability to be present for a vaccine.

The current vaccine program includes not only base line injections annually for influenza but now a separate set of base line injections for COVID with scheduled booster shots in the future for the base line and the variants of the virus. Such multiple injections for vaccines on a global scale are not efficient or effective from a manpower perspective.

To date, the distribution and administration of traditional injectable vaccines has been a time-consuming process from development to testing and then distribution. With rapidly mutating microbes, time is of the essence to stimulate the immune system in advance.

Often, side effects or reactions occur in response to material used in the carrier vehicles for the microbial materials. In some instance the side effects are long lasting and detrimental to overall health. The vaccines themselves often contain chemicals with current technologies that the human body has difficulty processing.

The invention claimed here solves the above problems.

### SUMMARY

In the present disclosure, a composition, method and system for antibody activation is provided.

Thus, by one broad aspect of the present invention, a composition is provided for antibody activation, the composition comprising an antigen and iodine.

By a further aspect of the present invention, a method is provided for preparing a composition for antibody activation, the method comprising providing an antigen, providing an iodine carrier for the antigen, and combining the antigen and the iodine carrier.

By a further aspect of the present invention, a system is provided for preparing a composition for antibody activation, the system comprising an airless reservoir to contain a volume of iodine, an injection device for injecting an antigen into the reservoir containing the iodine, and a pump for pumping the antigen and iodine mixture from the reservoir to a delivery device.

A further understanding of the functional and advantageous aspects of the invention can be realized by reference to the following detailed description and drawings.

### BRIEF DESCRIPTION OF THE DRAWINGS

Embodiments disclosed herein will be more fully understood from the following detailed description taken in connection with the accompanying drawings, which form a part of this application, and in which:

FIG. 1 is a schematic representation of a system for airless aqueous iodine production, according to an embodiment of the present disclosure.

FIG. 2 is a continuation of the schematic representation of FIG. 1, illustrating a bottling system for the airless aqueous iodine production system according to the embodiment of FIG. 1.

FIG. 3 is a schematic representation of an antigen being delivered with gaseous iodine, according to an embodiment of the present disclosure.

FIG. 4 is a schematic representation of an antigen being delivered with atmospheric iodine, according to a further embodiment of the present disclosure.

### DETAILED DESCRIPTION

The following description and drawings are illustrative of the disclosure and are not to be construed as limiting the disclosure. Numerous specific details are described to provide a thorough understanding of various embodiments of the present disclosure. However, in certain instances, well-known or conventional details are not described in order to provide a concise discussion of embodiments of the present disclosure.

Although the invention has been described with a preferred embodiment, it should be noted that the inventor can make various modifications, additions and alterations to the invention without departing from the original scope as described in the present disclosure.

The claimed invention is an antibody activator that differs and is an improvement from what currently exists. An improvement on the state of the art for vaccine production, safety and delivery is provided by combining viable inert pathogenic microbial materials together with iodine, for example a pure aqueous iodine solution.

In an aspect of the present invention, a composition for antibody activation is provided, the composition including an antigen and iodine.

The antigen is provided using inert virus or bacteria materials, which are readily available for historical pathogens and current pathogens including their variants when produced. The antigen is the material that activates the immune system through antibody recognition and binding. Antibody-antigen binding leads to amplification of an immune response.

Production of the antibody activator composition entails access to viable inert pathogenic microbial materials known to stimulate antibody production. Although theoretically it only takes one virus or bacteria to activate antibody production within the immune system, it is the invention's intent to provide sufficient antigenic material to enable maximum antibody production thereby affording maximum immune system protection.

Each product batch of the antibody activator can be directed at specific pathogens or solutions can contain multiple inert viral and bacteria microbial products for broad spectrum microbial introduction to the body.

The composition for antibody activation further includes iodine. Iodine is an essential micronutrient and has disinfection attributes that kill bacteria and deactivate viruses while leaving materials intact. Thus, the antigen is contained and delivered by way of a liquid essential micronutrient required daily by all humans.

In an embodiment of the composition, the iodine comprises a pure aqueous iodine solution.

In a preferred embodiment, the antigen is combined with pure aqueous iodine. Pure aqueous iodine is a stable medium to combine with antigenic viable inert virus and/or bacteria materials, to train the immune system to produce various antibodies concurrently.

A finished batch solution of known concentration and volume of aqueous iodine is produced and contained in an airless environment, capable of providing a stable environment for the inclusion and suspension of inert pathogenic microbial materials capable of activating the immune system to produce antibodies.

Use of aqueous iodine provides the ability to quickly and safely produce broad spectrum or targeted antibody activators in an oral spray. Iodine also kills microbes at the back of the throat, providing additional inert microbial materials to assist the antibody activation process. The product can alternatively be delivered by way of nasal spray or personal inhalation through nebulization. Delivery can be by spray or breathing, for example, a recipient can take five sprays at the back of the throat and swallow. The product can alternatively be sprayed onto skin for a topical antibody activator.

The composition allows for a daily use with controllable levels of microbial content being delivered in each dose, daily into the body system over extended periods of time, thereby allowing the immune system to gradually build antibody production and reduce stress on the immune system.

The composition improves on the state of the art logistics for vaccine production, safety and delivery by way of introducing viable inert pathogenic microbial materials suspended within a pure aqueous iodine solution. The suspension material is contained and delivered by way of a required liquid essential micronutrient that in-itself has disinfection capabilities and immune support attributes. Inert microbial material or “Natural Antibody Activators” suspended within the iodine solution are introduced by way of an oral spray for maximum absorption at safe levels for all ages. The inert microbial materials and the iodine dose are safely and effectively absorbed through the mucous membranes directly into the blood stream. The spray may also be used as a nasal or topical spray.

In an alternate embodiment of the composition, the iodine comprises a gaseous iodine.

In a further alternate embodiment of the composition, the iodine comprises an atmospheric iodine.

A method for preparing a composition for antibody activation is further provided, including providing an antigen and iodine, and combining the antigen and the iodine.

For the antigen, specific strains and quantities of viruses and or bacteria are selected and produced from approved level 4 laboratories. The viruses and bacteria are killed or deactivated by a solution of iodine within the level 4 laboratory. Selected inert microbial materials are then separated and shipped to an accredited laboratory, where the microbial material is combined with iodine.

In an embodiment of the method, providing the iodine comprises providing an airless aqueous iodine.

Pure aqueous iodine is produced and adjusted to specific concentrations to suspend the antigenic microbial material. A specific quantity of microbial materials is then introduced to a known volume of aqueous iodine under airless conditions to produce a stable finished product batch. The finished product is then filled into airless dispensing bottles fabricated from materials inert to iodine.

For small scale production, the live virus or viable bacteria can be introduced directly to the final batch aqueous iodine solution, thereby providing all the antigenic inert microbial materials.

In a further embodiment of the method, providing the iodine carrier comprises providing a gaseous iodine. A known quantity of solid iodine is utilized to produce gaseous iodine thereby eliminating the requirement of the water molecules within an aqueous iodine solution to act as the intermediary carrier vehicle.

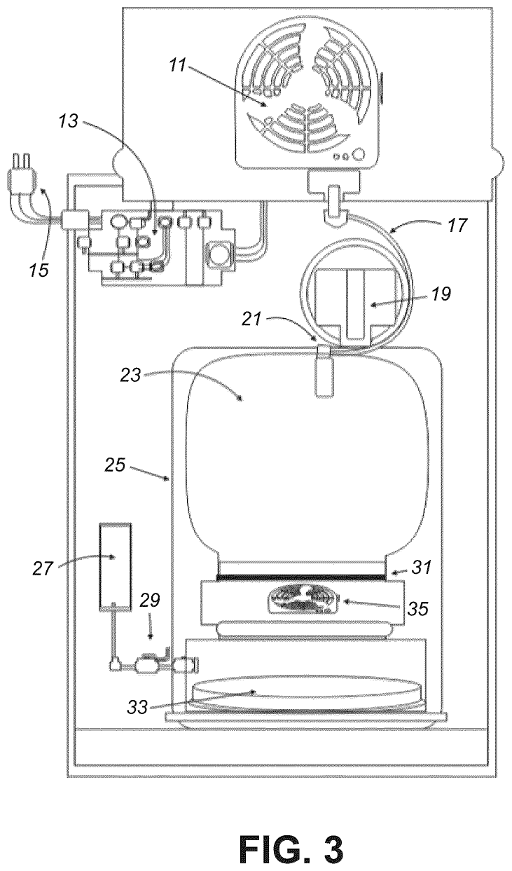

Referring to FIG. 3, solid iodine is introduced to a heated non sonic disk (heat plate) 33 to initiate, sustain and control the iodine sublimation process from a solid to gaseous iodine form. When the heat plate 33 reaches operating temperature, cooled iodine dispenser 27 dispenses solid iodine by activating valve 29, thereby introducing a known quantity of solid iodine to heat plate 33 and a known quantity of solid iodine is sublimed to a known quantity of gaseous iodine. Fan 35 may be activated, and iodine gas fills an iodine inert envelop 23 within an iodine containment cylinder 25. Mated airless connectors at the base 31 and apex 21 of iodine cylinder 25 are integral to the operation of the system.

Upon completion of the sublimation process, valve 29 is closed pending on fan 35 activation, and connector 31 is closed to seal iodine envelop 23. Peristaltic pump 19 is activated to accurately meter and draw iodine gas utilizing iodine inert hosing 17 from the iodine inert expandable envelop 23 through fan and dispersal ring 11 into a known environment or establishment.

In this embodiment, a cooling system is added to maintain the optimal environment for the solid iodine dispensing mechanism 27. The physical capturing and storing of the gaseous iodine is encompassed within the iodine cylinder 25. The airless expandable envelop 23, such as a teflon or kynar expandable envelop, within the iodine cylinder 25 acts as a gaseous iodine reservoir and is connected to the peristaltic micro-pump 19 that can accurately meter gaseous iodine from the iodine reservoir 23 and disperse it with the fan dispersal ring 11 into an environment or establishment. An electronic control system 13 controls one or more of the activating valve 29, fan 35, connectors 31, 21, peristaltic pump 19, and dispersal ring 11 and is powered through electric plug 15. This embodiment allows for both small and large scale production of gaseous iodine suited for commercial, medical, residential and industrial applications.

In a further embodiment of the method, providing the iodine carrier comprises providing an atmospheric iodine.

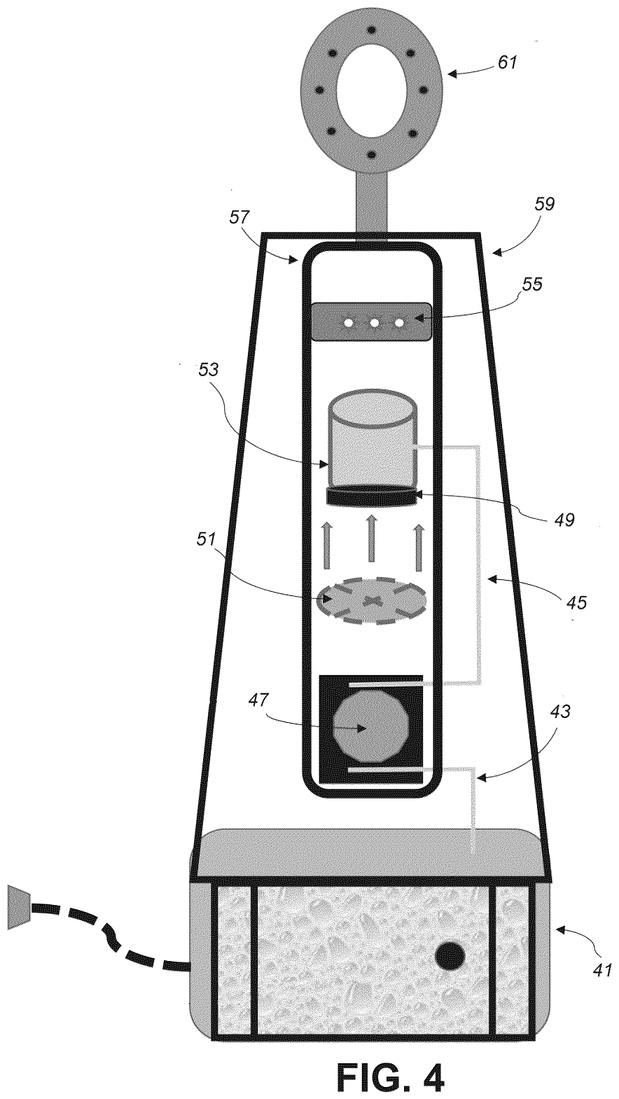

Referring to FIG. 4, atmospheric iodine is produced through a sublimation process of either liquid or solid iodine. The sublimation process is controlled by an electronic system 55 capable of monitoring and adjusting the function of devices within an apparatus. Gaseous iodine is directed to a dissemination fixture within the apparatus by way of positive directional airflow created by a fan 51. Atmospheric gaseous iodine is introduced in a controlled fashion into a given environment, for example a room or establishment, providing predetermined levels of atmospheric iodine per cubic meter within the environment.

A system for preparing a composition for antibody activation is also provided. The system includes an airless reservoir to contain a volume of iodine, an injection device for injecting an antigen into the reservoir containing the iodine, and a pump for pumping the antigen and iodine mixture from the reservoir to a delivery device.

In an embodiment of the system, the system includes an airless aqueous iodine production system, and the delivery device comprises an airless spray bottle.

Specific known quantities of inert pathogenic materials are selected and introduced and suspended with the iodine solution. Pure aqueous iodine being non-invasive or destructive to inert microbial materials is an ideal carrier solution. Although aqueous iodine is volatile, it can be contained and controlled through airless bottle dispensing technologies.

The microbial materials are injected into a known concentration and volume of pure aqueous iodine from 5 parts per billion to 300 parts per million to produce a finished product batch contained within the airless reservoir environment.

The finished product of antibody activators are then pumped from the batch reservoir and injected into airless spray bottles, ready for packaging and distribution.

The antibody activators are delivered in an airless spray bottle ensuring product integrity and viability. Alternatively, but not desirable due to shelf life issues and stability, the antibody activators can be delivered in glass bottles with sprayers. This delivery device allows shipping direct to the recipient for simple administration. Alternatively the antibody activators solution can be delivered in a dropper format for oral ingestion.

A preferred delivery device is an airless bag in bottle design with a volume of 5 ml to 30 ml for example with a teflon or like material insert bag, inert to iodine, which provides a stable environment and shelf-life for the antibody activators and aqueous iodine solution. A micro-dosing spray mechanism delivery system for oral, nasal or topical applications provides accurate product dosing in each spray.

Antibody activators within the iodine solution are introduced by way of an oral, nasal or topical spray for maximum absorption at safe levels. The inert microbial materials and the iodine dose are safely and effectively absorbed through the mucous membranes directly into the blood stream, or concurrently into the gut when swallowed post spray administration or delivered in dropper format.

As illustrated in FIG. 3, in an alternate embodiment of the system, the system includes a gaseous iodine production system and the delivery device comprises a fan and a dispersal ring 11. In this embodiment, a cooling system maintains an optimal environment for a solid iodine dispensing mechanism 27. Solid iodine is introduced to a heated non sonic disk (heat plate) 33 to initiate, sustain and control the iodine sublimation process from a solid to gaseous iodine form. When the heat plate 33 reaches operating temperature, cooled iodine dispenser then dispenses solid iodine by activating valve 29 introducing a known quantity of solid iodine to heat plate 33 and a known quantity of solid iodine is sublimed to a known quantity of gaseous iodine. Once the iodine gas is produced, fan 35 may be activated and the iodine gas fills an iodine inert envelop 23 that acts as an iodine reservoir within iodine containment cylinder 25, for example an airless teflon or kynar expandable envelop. Upon completion of the sublimation process, valve 29 and connector 31 are closed to seal iodine envelop 23. Peristaltic pump 19 connects iodine envelop 23 to fan and dispersal ring 11 through an iodine inert hosing 17, to accurately meter iodine gas through fan and dispersal ring 11 into a known environment or establishment. An electronic control system 13 controls the iodine dispension and is powered through an electric plug 15. This embodiment allows for both small and large scale production of gaseous iodine by the invention suited for commercial, medical, residential and industrial applications.

Referring again to FIG. 4, a further embodiment of the system includes an atmospheric iodine production system. Atmospheric iodine is produced through a sublimation process of either liquid or solid iodine. The sublimation process is controlled by an electronic system 55 capable of monitoring and adjusting the function of devices.

Gaseous iodine is directed by a peristaltic pump 47 from a replaceable iodine canister 41 through liquid feed supply line 43 connected to the pump 47. The pump 47 then meters liquid through feed line 45 to supply airless iodine reservoir 53. The gaseous iodine is then directed to a dissemination fixture (delivery device) 61 by way of positive directional airflow created by a fan 51. The fan 51 is activated concurrently to a sonic disk 49 activation. Upon sonic disk 49 activation, gaseous iodine is produced from the airless iodine reservoir 53 and exits the apparatus through the dispersal ring 61 in a room or establishment. Atmospheric gaseous iodine is introduced through the perforated gaseous iodine dispersal ring 61 in a controlled fashion into a given environment, for example a room or establishment, providing predetermined levels of atmospheric iodine per cubic meter within the environment. A controller 55 may be programmed to accommodate room dimensions and air turnover within said room. Component housing spire 57 encloses the system.

A system for airless aqueous iodine production is provided in more detail herein. The system provides precise aqueous iodine production and dosing with inline assessment and automated regulation of iodine content. The system further provides containment and bottling of finished batch aqueous iodine to maintain the iodine concentration. The system can be used with injection of the antigenic material into the finished pure aqueous iodine solution for production of the antibody activators.

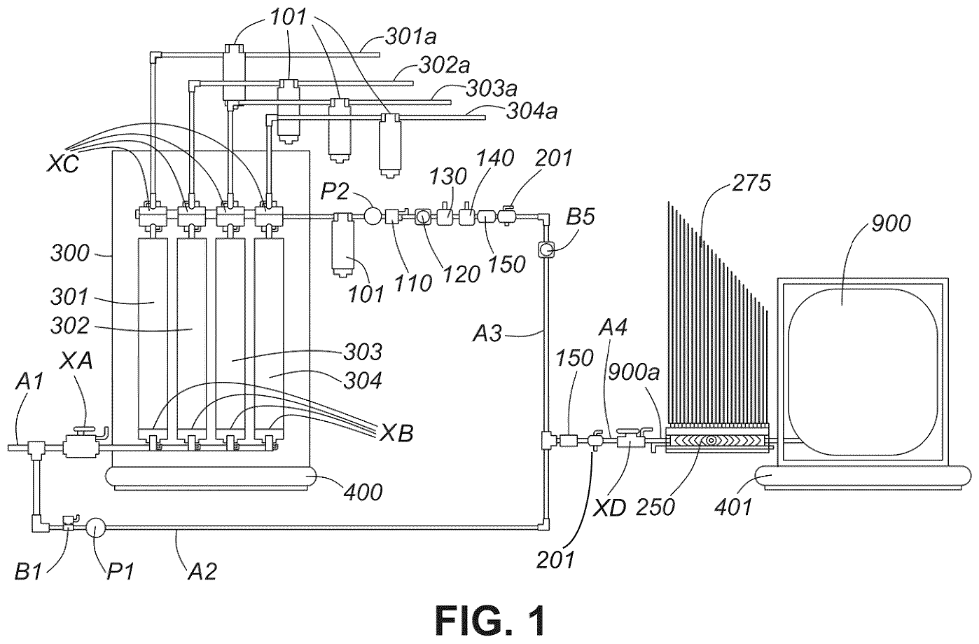

Referring again to FIG. 1, an embodiment of the system for airless aqueous iodine production is shown and includes a plurality of iodine columns 301, 302, 303, 304 to hold iodine. A water feed line A1, connected through a water feed line valve XA, provides water to the iodine columns 301, 302, 303, 304. Additional iodine column valves XB control the water flow for each iodine column 301, 302, 303, 304. A water by-pass line A2 diverts water away from the water feed line A1, bypassing the iodine columns 301, 302, 303, 304, to connect and supply water to a finished aqueous iodine supply line A4, described below.

An iodine concentrate supply line A3 is connected to the iodine columns 301, 302, 303, 304 through an iodine concentrate supply line valve XC, and carries iodine concentrate from the iodine columns 301, 302, 303, 304 to the finished aqueous iodine supply line A4. The iodine concentrate supply line A3 includes a sensor 110, 120 for measuring the iodine concentration in the iodine concentrate.

Both the iodine concentrate supply line A3 and the water by-pass line A2 connect to a first end of the finished aqueous iodine supply line A4. An inline mixer 150 in the finished aqueous iodine supply line A4 mixes the iodine concentrate with the water from the water bypass line A2 to produce a finished aqueous iodine.

A second end of the finished aqueous iodine supply line A4 is connected to a manifold 250 through a finished aqueous iodine supply line valve XD. The other end of the manifold 250 is connected to an airless iodine product reservoir 900, for storing the airless aqueous iodine. A vacuum system 900a connected to the manifold 250 removes air from the iodine product reservoir 900.

A programmable logic controller 999 is connected to the water feed line valve XA, the iodine column valves XB, the iodine concentrate supply line valve XC and the sensor 110, 120, and controls the valves XA, XB, XC in response to measurements from the sensor 110, 120, thereby regulating the production and concentration of the finished aqueous iodine.

In an embodiment, an iodine generator box 300 supports the iodine columns 301, 302, 303, 304. An iodine generator scale 400 is connected to the iodine generator box 300, and measures the weight of the iodine in the iodine columns 301, 302, 303, 304. The weight measurement from the iodine generator scale 400 is provided to the programmable logic controller 999.

In an embodiment, an airless aqueous iodine scale 401 is connected to the airless iodine product reservoir 900, and measures the weight of the airless aqueous iodine and provides the measurement to the programmable logic controller 999.

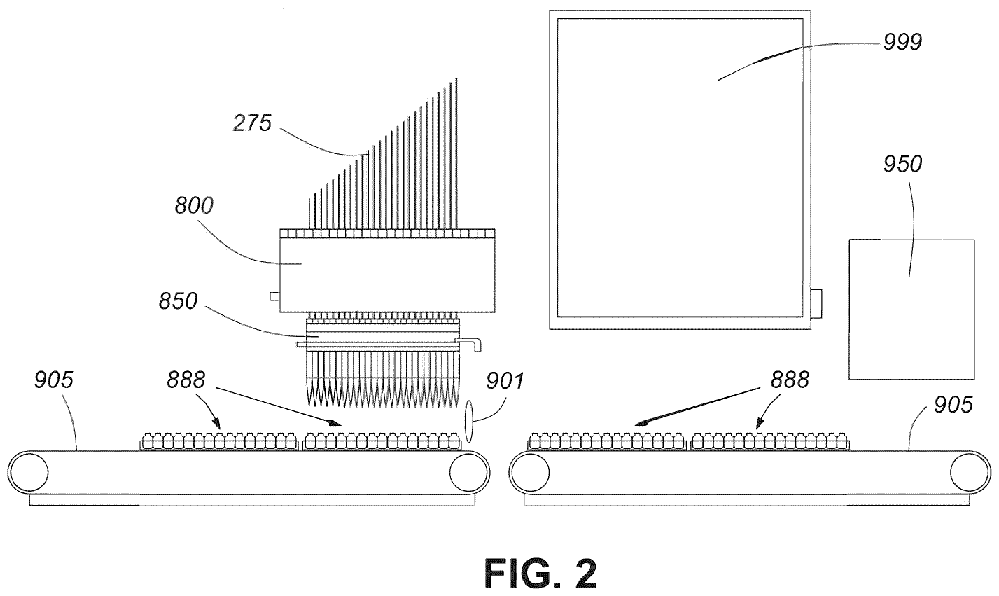

Referring to FIG. 1 and FIG. 2, in a further embodiment, the system includes manifold exit ports (not shown) and transfer pump lines 275, each transfer pump line 275 connected to one of the manifold exit ports. Fill pumps 800 are connected to the transfer pump lines 275 and move airless aqueous iodine from the airless iodine product reservoir 900 through the transfer pump lines 275. Bottles are moved in a fitted jig 888 on a conveyor belt 905 to the transfer pump lines 275 for filling, and a fill line plate system 850 positions the bottles on the conveyor 905 to be filled by the transfer pump lines 275.

In a further embodiment, the system includes a flow sensor/valve B1 on the water bypass line A2. The flow measurements of the flow sensor/valve B1 are sent to the programmable logic controller 999 to regulate a volume controller of water feed line A1 and water bypass line A2. The system further includes a flow sensor B5 on the iodine concentrate supply line A3. The flow measurements of the flow sensor B5 are sent to the programmable logic controller 999 to regulate a volume controller of the water bypass line A2 to the finished aqueous iodine supply line A4.

In a further embodiment, the system includes more than one iodine concentrate supply line 301a, 302a, 303a, 304a, each iodine concentrate supply line connected through an iodine concentrate supply line valve XB to one of the iodine columns 301, 302, 303, 304. This allows for multiple concentrations or batches of iodine concentrate to be produced in parallel.

The sensor 110, 120 for measuring iodine concentration in the aqueous iodine production system may be an in-line electronic sensor and/or an in-line spectrophotometer.

A method for producing airless aqueous iodine includes providing water through a water feed line A1 to a plurality of iodine columns 301, 302, 303, 304 and flowing water through the plurality of iodine columns to produce an iodine concentrate. The iodine concentrate is passed from the iodine columns 301, 302, 303, 304 through an iodine concentrate supply line A3 and filtered through a sediment filter 101. The iodine concentration of the concentrate is adjusted with water from a water bypass line A2 in a finished aqueous iodine supply line A4 to produce an aqueous iodine. The aqueous iodine is passed through a manifold 250 that includes a vacuum system 900a. The vacuum system 900a removes air from the iodine product reservoir 900 while supply line valve XD is shut, thereby providing the airless iodine product reservoir 900. Supply line valve XD is opened to fill the airless iodine product reservoir 900 with the aqueous iodine for storage.

In an embodiment of the method, the iodine concentration is measured in the iodine concentrate supply line using a sensor 110, 120. The sensor 110, 120 is an electronic sensor and/or a spectrophotometer.

The flow of the water through the water feed line A1 is regulated by a water feed line valve XA. The flow of the iodine concentrate through the iodine concentrate supply line A3 is regulated by an iodine concentrate supply line valve XC. The water flow, iodine concentrate flow, and the iodine concentration measurements are received by a programmable logic controller 999. In response, the programmable logic controller 999 controls the water feed line valve XA and the iodine concentrate supply line valve XC to adjust the iodine concentration of the iodine concentrate.

The flow of the water through the water bypass line A2 is measured by a flow sensor/valve B1, connected to the programmable logic controller 999, which in turn regulates flow through the water feed line A1 and the water bypass line A2 through a volume regulator. The flow of the iodine concentrate through the iodine concentrate supply line A3 is measured through a flow sensor B5, connected to the programmable logic controller 999, which in turn regulates flow through the water bypass line A2 and finished aqueous iodine supply line A4 through a volume regulator. In this way, flow of water through the iodine columns 301, 302, 303, 304 is regulated to achieve a target iodine concentration in the iodine concentrate, and to further adjust the iodine concentration of the finished aqueous iodine with water from the water bypass line A2.

In a further embodiment of the method, measuring the iodine concentration includes measuring a weight of the iodine columns 301, 302, 303, 304 using an iodine generator scale 400 connected to an iodine generator box 300 supporting the iodine columns, measuring a weight of the airless aqueous iodine using an airless aqueous iodine scale 401 connected to the airless iodine product reservoir 900, providing the iodine column weight and the airless aqueous iodine weight to the programmable logic controller 999, and calculating the iodine concentration of the airless aqueous iodine by the programmable logic controller using the iodine column weight and the airless aqueous iodine weight.

In a further embodiment, the airless aqueous iodine is drawn from the reservoir 900 through transfer pump lines 275 using a fill pump 800, and from the transfer pump lines 275 to fill bottles with the airless aqueous iodine. The bottles are in a jig 888 provided by a conveyor belt 905 and positioned for filling by a fill line plate system 850.

In an embodiment, multiple iodine concentrate supply lines 301a, 302a 303a, 304a are included, each iodine concentrate supply line connected to one iodine column 301, 302, 303, 304, thereby allowing production of a plurality of concentrations of iodine concentrate.

The production of aqueous iodine using the system may be carried out in more than one location. For example, the iodine columns 301, 302 303, 304 may be in one location, the airless iodine product reservoir 900 may be in another location, and the bottling of the airless aqueous iodine on a conveyor 905 may be in another location.

In a further embodiment, the pH of the iodine concentrate is adjusted using a base injection port 130 and an acid injection port 140 in the iodine concentrate supply line A3. A sample of iodine concentrate may also be removed using a sample port 201 in the iodine concentrate supply line A3.

The present invention provides three different mechanical methods within the iodine production system to accurately assess a known quantity of aqueous iodine being produced and in a finished batch, regardless of environmental conditions. This system also provides unique high flow refillable iodine columns 301, 302 303, 304 within an iodine generator apparatus that can be programmed to produce specific batch lots and species of iodine. This iodine system generator can accommodate different forms and quality of solid iodine concurrently. This iodine generator has vertical iodine columns 301, 302 303, 304 running in parallel allowing for the highest output of aqueous iodine production. This iodine system has a scalable, iodine generator apparatus. An automated programmable logic controller 999 is programmed to operate the complete iodine production and bottling invention and control input water to the iodine apparatus to produce aqueous iodine ready for bottling. The finished aqueous iodine batch lot is produced and is accumulated in an airless reservoir 900 to control the integrity of the finished product. The automated system then activates the bottling component including peristaltic pumps with iodine resistant fill lines to maintain the integrity of the finished aqueous iodine product. A 16 kilogram Iodine generator apparatus as described is capable of bottling in excess of 200,000 bottles of precisely controlled formulated aqueous iodine per hour.

The present invention provides for high flow multiple paralleled iodine columns 301, 302 303, 304, within a pure aqueous iodine generation apparatus, comprising several inline iodine content monitoring systems coupled with unique airless mixing and finished formula aqueous iodine batch reservoirs; capable of sustaining finished batches of aqueous iodine for prolonged periods of time. Unique stackable airless finished batch iodine product reservoirs 900 are directly connected to iodine bottling systems comprising peristaltic pumps with iodine inert fill hoses to maintain product integrity of an airless iodine generation system to the bottling fill point. Airless, traditional glass or plastic bottles are placed and filled within custom acrylic jigs 888 to accommodate the iodine fill lines. The iodine fill lines are located within a horizontal mobile acrylic or the like, plate apparatus 850 accepting the iodine bottle fill lines. This fill line plate 850 is mounted horizontally above a programmed controlled conveyor belt 905 that moves the bottles within the acrylic jigs 888 to mate with the iodine fill lines. This entire process from aqueous iodine production, to finished bottled product is orchestrated by a programmable logic controller 999 with specific input codes to produce and bottle aqueous iodine at predetermined precise programmed formulas without human intervention. This invention does allow for human iodine sampling and intervention within the programming. This system is not affected by environmental conditions, water temperature, or water flow rate.

The present invention is fully automated. Its valved directional water flow dynamics, inline iodine monitoring systems comprising spectrophotometry, electrical current feedback and weights and measures, allows for pure accurate aqueous iodine production and iodine content management within a pure aqueous iodine solution from generation to finished aqueous iodine product batches and lots. This invention responds instantly to environmental changes to ensure aqueous iodine content is known and accurate at all times. This invention provides for a total airless aqueous iodine system from production to the end bottling point ensuring product integrity throughout the process. The embodiment of paralleled sixteen kilogram iodine generation columns invention allows high flow aqueous iodine production, accepting different types, forms and quality of solid iodine to produce aqueous iodine independently in each column or a combination variation of product can be produced. This invention allows for in excess of 30 different aqueous iodine formulations to be produced and bottled simultaneously. The aqueous iodine species and formulations of aqueous iodine generated may be varied and controlled through adjustment product injection points at specific locations within the system. This iodine production and bottling system invention is capable of bottling in excess of 200,000 bottles an hour, per bottling line, with increased expansion capabilities. All the processes are implemented by the programable logic controller.

An example embodiment is presented below:

Referring again to FIG. 1, the water flight path starts at A1 and splits. One line, the water feed line A1, enters iodine generator box 300, while the second line, water bypass line A2, bypasses the system for a re-blend if necessary. This process is controlled by a solenoid valve, water feedline valve XA. Water enters the iodine generator box 300 and flows through iodine columns 301, 302, 303, 304 in contact with the iodine within the columns. Iodine concentrate supply line valves XB at the top of the iodine columns control the water direction after passing through the iodine columns 301, 302 303, 304. Aqueous iodine concentrate now passes through iodine concentrate supply line A3 and enters a 1 micron filter 101, then passes by an electronic sensor 110 providing an iodine electronic reading converted to content value, then passes through a photo-spectrometry lens 120 to gauge the colour metrics of the solution and create a content value. Then either a base 130 or an acid 140 can be injected into the iodine concentrate, and the iodine concentrate passes through an inline helicoil mixer 150, then a sample port 201, and passes by flow sensor B5 to provide a value. The iodine concentrate then blends with water from water bypass line A2 through a helicoil mixer 150 to create finished the finished formula in aqueous iodine supply line A4. The finished aqueous iodine then passes through sample calibration port 201, followed by Solenoid supply line valve XD, then enters the manifold 250 fitted with exit ports for the transfer pump lines 275 with 900a vacuum system for Airless Aqueous Iodine reservoir 900. Upon filling the reservoir 900, scales 400 and 401 evaluate the content of iodine used from iodine generator box 300 and Airless Aqueous Iodine reservoir 900. All Data Inputs are relayed to programmable logic controller 999 to operate the system and produce predetermined finished formula batches by direct aqueous iodine production or a re-blend with line A2 for adjustments.

Referring to FIG. 2, at this point the programmable logic controller 999 activates and primes fill pumps 800, drawing from batch reservoir 900 to start the bottling process. Bottles are place in a fitted jig 888, and are transported along conveyor belt 905 to a precise filling point on the line determined by light sensor 901. Fill line plate system 850 is pneumatically operated and positioned to fill bottles within jig 888 on conveyor belt 905. Once bottles are filled, the jigs 888 continue on to traditional bottle capping and labeling 950.

Each of the valves within the system controls water direction and flow rates to either enter the iodine generator apparatus, blend it or bypass it. The iodine generator is controlled by multidirectional valves and consists of four columns 301a, 302a, 303a, 304a of iodine that can operate independently or in unison to produce various blends and concentrations of iodine providing different formula outcomes simultaneously. The aqueous iodine solution is verified during production by way of electrical iodine sensing 110, spectrophotometry 120, and weights and measure 400, 401 in the final product. It can also be sampled and tested manually. The final product is held in an airless vessel 900 and then a peristaltic pumps system 800 delivers the aqueous formula from the airless vessel to the liquid bottling line to fill a predetermined volume bottle. On the bottling line special jigs 888 retain the bottles and are transported along a conveyor belt 905 to the precise point of filling. At that time a fill plate containing multiple fill lines is lowered pneumatically to fill the bottles in the jig. The fill plate 850 also contains ventilation to remove any gaseous iodine from the bottling line during production. The bottles are then capped, labelled and boxed to be shipped.

Electronic valves, flow sensors, pressure gauges, electrical iodine readings, photo light readings, weights and measures provide information to the programmable logic controller 999 to accurately produce a predetermined formula by controlling water flows and blending ratios based on actual input values from the sensor arrays within the apparatus.

All of the components to assemble this invention can be purchased. Prior to introducing the programmable logic controller 999 and the online electronic components, start by building the system with manual valves and human sampling ports for product content and verification. Ensure the system is pressurized and airless through to the final end in the aqueous iodine reservoir 900. Utilize peristaltic pumps connected to the airless aqueous iodine reservoirs to bottle an aqueous iodine formula. Once the system is operational and tested, replace the manual valves with appropriate solenoid valves as per the invention. Add the inline iodine content sensing 110, 120 and detection systems, then connect to the programmable logic controller 999 to complete the invention as described.

In an alternative version of the invention, a booster pump on water feed line A1 may be added to account for pressure drops across the iodine generator apparatus to allow for positive pressure blending of water feed line A1 and water bypass line A2 within the overall system. An additional iodine generator apparatus can be added to increase production rates, or additional iodine columns can be added to the iodine column apparatus to increase aqueous iodine production within the overall system.

Another alternative version is the single source water feed line A1 listed in the invention, may also be provided from two separate source water supplies. Iodine generation, aqueous iodine storage and iodine bottling may be done in separate locations. This invention provides for an in system blending of the final product, alternatively the final airless aqueous iodine reservoirs may also be utilized for blending a final product. The iodine generator columns may alternatively be structured in a “series” water flow configuration, but not recommended. The airless iodine reservoirs 900 may also be stackable. The airless iodine reservoirs 900 can also be used to ship or store finished or raw iodine concentrate product for extended periods of time.

With this invention, in excess of thirty different aqueous iodine derivative products can be produced simultaneously, onsite, in any country around the world; for humanitarian, medical, disease, drug applications and more can be produced accurately, inexpensively and in large quantities by this invention. Pure Aqueous Iodine disinfection products, human and animal micronutrients, drugs, agriculture products and more can be made available.

Additionally, this invention is designed to generate and produce a bottle of precise iodine content and formula. The design of the iodine generator apparatus within the invention allows for high production rates and long retention times of pure aqueous iodine for alternative product applications. such as spraying crops and soil, disinfecting livestock drinking water, filling large and small containers with aqueous iodine for various applications.

Also, this invention can alter the biocidal species of aqueous iodine. This invention can convert iodine to its ionic state. This invention can produce multiple iodine product formulas concurrently.

The composition, method and system for antibody activators provides a simple, safe oral, nasal or topical spray, that can be administered personally to train the immune system to make antibodies anywhere anytime. Having this invention made available to the public would reduce the strain on the global medical system and provide a personal solution that can be delivered to a person's residence.

This invention also applies and provides protection within the animal kingdom. Antibody activator solution can be produced specifically for targeted or broad-spectrum microbial issues and dispensed in aqueous form or aerosolized for diseases within but not limited to the pet industries, commercial livestock and poultry industries.

The present invention has been shown and described in a preferred embodiment. It is recognized, however, that departures may be made within the scope of the invention and that obvious modifications will occur to a person skilled in the art. With respect to the above description, it is to be realized that the optimum dimensional relationships for the parts of the presented invention, to include variations in size, materials, shape, form, function, and manner of operation, assembly and use, are deemed readily apparent and obvious to one skilled in the art, and all equivalent relationships to those illustrated in the drawings and described in the specifications are intended to be encompassed by the present invention.

What is claimed is:

1. A composition for antibody activation, the composition comprising:
an antigen; and
iodine.

2. The composition of claim 1, wherein the iodine comprises a solution wherein said iodine solution is a pure aqueous iodine solution.

3. The composition of claim 1, wherein the iodine comprises a solution where said iodine solution is a gaseous iodine.

4. The composition of claim 1, wherein the iodine comprises a solution where said iodine solution is an atmospheric iodine.

5. A method for preparing a composition for antibody activation, comprising:
providing an antigen;
providing an iodine carrier for the antigen; and
combining the antigen with the iodine carrier.

6. The method of claim 5, wherein providing the iodine carrier comprises providing an airless aqueous iodine.

7. The method of claim 6, further wherein providing the airless aqueous iodine comprises:
providing water through a water feed line to a plurality of iodine columns;
flowing water through the plurality of iodine columns to produce an iodine concentrate;
passing the iodine concentrate through an iodine concentrate supply line;
filtering the iodine concentrate through a filter;
measuring an iodine concentration of the iodine concentrate using a sensor;
adjusting the iodine concentration of the iodine concentrate with water from a water bypass line to produce an aqueous iodine;
passing the aqueous iodine through a manifold comprising a vacuum system to an airless iodine product reservoir for storage of an airless aqueous iodine.

8. The method of claim 5, wherein providing the iodine carrier comprises providing a gaseous iodine.

9. The method of claim 5, wherein providing the iodine carrier comprises providing an atmospheric iodine.

10. A system for preparing a composition for antibody activation, the system comprising:
an airless reservoir to contain a volume of iodine;
an injection device for injecting an antigen into the reservoir containing the iodine; and
a pump for pumping mixture of the antigen and the iodine from the reservoir to a delivery device.

11. The system of claim 10, further comprising:
a plurality of iodine columns to hold iodine;
a water feed line connected through a water feed line valve to the iodine columns, for providing water to the iodine columns;
a water by-pass line having a first end and a second end,
the first end diverting from the water feed line, for diverting water away from the water feed line; and
the second end connected to a finished aqueous iodine supply line, for supplying water to the finished aqueous iodine supply line;
at least one iodine concentrate supply line connected through an iodine concentrate supply line valve to at least one of the iodine columns, for directing iodine concentrate from the at least one iodine column to the finished aqueous iodine supply line, and comprising a sensor for measuring iodine concentration;
the finished aqueous iodine supply line comprising:
a first end connected to the iodine concentrate supply line and a water bypass line;
an inline mixer for mixing the iodine concentrate with the water from the water bypass line to produce a finished aqueous iodine; and
a second end;
a manifold having
a first end connected to the second end of the finished aqueous iodine supply line through a finished aqueous iodine supply line valve;
a second end connected to an iodine product reservoir, for storing airless aqueous iodine; and
a vacuum system for removing air from the iodine product reservoir;
a programmable logic controller connected to the water feed line valve, the iodine concentrate supply line valve, and the sensor, for controlling the valves in response to measurements from the sensor;
for producing a pure airless aqueous iodine; and
further wherein the delivery device is an airless spray bottle.

12. The system of claim 10, further comprising:
a cooled iodine dispenser for dispensing solid iodine;
a heat plate for receiving and sublimating the solid iodine from the dispenser; and
a fan for directing sublimated gaseous iodine to the airless reservoir;
and wherein the delivery device comprises a dispersal ring for directing the gaseous iodine to an environment.

13. The system of claim 10, wherein
the reservoir comprises a heated sonic reservoir for receiving airless liquid iodine and producing atmospheric humidified iodine-enriched air and gaseous iodine; and
the delivery device comprises a perforated gaseous iodine dispersal ring for providing atmospheric iodine.
